(19) United States
(12) Patent Application Publication
(10) Pub. No.: US 2025/0352100 A1
(43) Pub. Date: Nov. 20, 2025

(54) FLUID SAMPLING APPARATUS, METHOD AND KIT

(71) Applicant: Volumeer AB, Stockholm (SE)

(72) Inventors: Mehmet Volkan Özenci, Stockholm (SE); Jan Patrick Klingler, Stockholm (SE)

(21) Appl. No.: 19/119,991

(22) PCT Filed: Oct. 4, 2023
(86) PCT No.: PCT/SE2023/050988

§ 371 Date: Apr. 10, 2025

(30) Foreign Application Priority Data
Oct. 12, 2022 (GB) 2215054.4

(51) Int. Cl. A61B 5/15

(52) U.S. Cl. CPC A61B 5/150236; A61B 5/150221; A61B 5/150267; A61B 5/150351; A61B 5/15074

(57) ABSTRACT

A fluid sampling apparatus comprising: a primary fluid chamber configured to receive a fluid therein, the primary fluid chamber defined by a barrel and a plunger. Movement of the plunger through the barrel varies the volume of the primary fluid chamber. A first end of the barrel comprises a fluid inlet port which is couplable to a fluid inlet tube. The fluid inlet port includes a first one-way valve. The plunger comprises a plunger channel therethrough including a second one-way valve. The plunger channel and second one-way valve are configured to provide for fluid communication between the primary fluid chamber and a second end of the plunger. The second end of the plunger is configured to engage with a sample vessel for receiving a fluid from the plunger channel.

### FIELD

The present disclosure relates to a fluid sampling apparatus. In particular, the present disclosure relates to a fluid sampling apparatus which may be used for sampling a bodily fluid such as blood, a method of using a fluid sampling apparatus to sample a fluid and a kit of parts comprising a fluid sampling apparatus.

### BACKGROUND

Acquiring bodily fluids from a patient is a deceptively difficult process which is complicated by several factors. A few examples are provided below.

Firstly, initial fluid drawn from a patient tends to comprise impurities and contaminants which make such initial fluid unsuitable for many types of tests. These impurities and contaminants may come from, for example, dirt on the skin or even small amounts of the skin itself where a needle pierced the body. As such, it is desirable to dispose of, or at least separate, an initial fluid sample prior to acquiring a sample suitable for testing or analysis.

Secondly, it can be a challenge to obtain fluid samples that have a consistent volume. Certain tests and procedures require particular volumes of fluid in order to be effective. As a result of this, where a sample is obtained that is of an insufficient volume, an entirely new sample may need to be drawn from a patient, thereby wasting time, patient fluid, sample bottles, needles and increasing discomfort for the patient.

### SUMMARY

According to a first aspect of the present disclosure, there is provided a fluid sampling apparatus comprising: a primary fluid chamber configured to receive a fluid therein, the primary fluid chamber defined by a barrel and a plunger, wherein the plunger is arranged at least partially within the barrel such that the movement of the plunger through the barrel varies the volume of the primary fluid chamber, wherein: a first end of the barrel comprises a fluid inlet port wherein the fluid inlet port is couplable to a fluid inlet tube, wherein the fluid inlet port includes a first one-way valve and wherein the fluid inlet port and the first one-way valve are configured to provide for fluid communication between a coupled fluid inlet tube and the primary fluid chamber; the plunger comprises a plunger channel therethrough including a second one-way valve, the plunger channel and second one-way valve are configured to provide for fluid communication between the primary fluid chamber at a first end of the plunger and a second end of the plunger wherein the first end of the plunger defines an inner surface of the primary fluid chamber and the second end of the plunger opposes the first end of the plunger; and wherein the second end of the plunger is configured to engage with a sample vessel for receiving a fluid from the plunger channel.

In one or more embodiments, the second end of the plunger may be configured to be interchangeably couplable between each of an initial vessel and a sample vessel. The initial vessel may comprise a different cross-section to the sample vessel. The initial vessel may comprise a different cross-sectional shape to the sample vessel. The initial vessel may comprise a different cross-sectional area to the sample vessel. The initial vessel may comprise a different cross-sectional diameter to the sample vessel. The cross-section referred to is that of the opening of the initial vessel and sample vessel.

In one or more embodiments, the plunger may be moveable between a first position and a second position wherein, in the first position, the fluid inlet port is in fluid communication with the plunger channel such that fluid does not flow into the primary fluid chamber and in the second position, the primary fluid chamber has a predefined volume and the fluid inlet port is in fluid communication with the primary fluid chamber such that fluid flowing into the fluid inlet port enters the primary fluid chamber.

In one or more embodiments, the fluid sampling apparatus may comprise a sample vessel adapter arranged at the second end of the plunger configured to at least partially engage with the sample vessel when the sample vessel is fluidly coupled to the primary fluid chamber via the plunger channel.

In one or more embodiments, the second end of the plunger may further comprise a piercing member which is configured to pierce a cap of a sample vessel to direct the flow of fluid from the plunger channel to the sample vessel.

In one or more embodiments, the fluid sampling apparatus may comprise an actuation arm coupled at a first end of the actuation arm to the barrel and wherein a second, opposite, end of the actuation arm extends beyond the second end of the sample vessel adapter when the plunger is in the first position and when the plunger is in the second position, the second end of the actuation arm is in line with the second end of the sample vessel adapter.

In one or more embodiments, the second end of the actuation arm may comprise a load bearing surface at the second end of the actuation arm wherein the load bearing surface extends perpendicularly to the length of the actuation arm such that the load bearing surface is configured to provide a surface against which force can be distributed in order to reduce a pressure on a surface pressing against the second end of the actuation arm.

In one or more embodiments, the fluid sampling apparatus may further comprise an initial vessel retention arm wherein the initial vessel retention arm is movable between a first position and a second position wherein the initial vessel retention arm comprises an initial vessel retention aperture in which an initial vessel can be retained and wherein: when the initial vessel retention arm is in the first position, at least a portion of the initial vessel retention arm is configured to substantially align the initial vessel retention aperture with the piercing member such that an initial vessel retained within the initial vessel retention aperture is pierced by the piercing member and when the initial vessel retention arm is in the second position, the initial vessel retention aperture is not in line with the piercing member such that the initial vessel retention arm does not impede the coupling of a sample vessel to the piercing member.

In one or more embodiments, the initial vessel retention arm may comprise: a first initial vessel retention sub-arm, wherein the first initial vessel retentions sub-arm is coupled to one of the barrel, the plunger or the sample vessel adapter; and a second initial vessel retention sub-arm wherein the second initial vessel retentions sub-arm comprises the initial vessel retention aperture, and wherein the first initial vessel retention sub-arm is coupled to the second initial vessel retention sub-arm by an articulated joint and wherein, in its first position, the second initial vessel retention sub-arm extends perpendicularly to the first initial vessel retention sub-arm.

In one or more embodiments, when in the second position, the second initial vessel retention sub-arm may extend parallelly to the first initial vessel retention sub-arm

In one or more embodiments, the first initial vessel retention sub-arm may be coupled to the barrel and the initial vessel retention sub-arm is the actuation arm.

In one or more embodiments, the actuation arm may be movable between a first position and a second position wherein the actuation arm comprises an initial vessel retention aperture in which an initial vessel can be retained and wherein: when the actuation arm is in the first position, at least a portion of the actuation arm is configured to substantially align the initial vessel retention aperture with the piercing member such that an initial vessel retained within the initial vessel retention aperture is pierced by the piercing member and when the actuation arm is in the second position, the actuation arm is not in line with the piercing member such that the actuation arm does not impede the coupling of a sample vessel to the piercing member.

According to a second aspect of the present disclosure, there is provided a method of extracting a fluid using a fluid sampling apparatus of the first aspect, the method comprising: coupling the fluid inlet port to a fluid inlet tube at a second end of the fluid inlet tube and wherein the fluid inlet tube is configured to receive a fluid at a first end of the fluid inlet tube; coupling an initial vessel to the second end of the plunger such that fluid flows from the fluid inlet tube through the fluid sampling apparatus via the first and second one-way valves and into the initial vessel; removing the initial vessel;

moving the plunger within the barrel such that the volume of the primary fluid chamber is increased, thereby creating a negative pressure in the primary fluid chamber and causing fluid to flow from the fluid inlet tube through the first one-way valve into the primary fluid chamber; coupling a sample vessel to the second end of the plunger such that fluid flows from the primary fluid chamber into the sample vessel via the second fluid inlet valve.

In one or more embodiments, moving the plunger within the barrel such that the volume of the primary fluid chamber is increased may comprise moving the barrel from the first position to the second position.

In one or more embodiments, the step of coupling an initial vessel to the second end of the plunger may comprise moving the initial vessel retention arm into the first position and retaining the initial vessel within the initial vessel retention aperture; and the step of removing the initial vessel comprises removing the initial vessel retention arm from the initial vessel retention aperture and moving the initial vessel retention arm into the second position.

According to a third aspect of the present disclosure, there is provided a kit of parts comprising a fluid sampling apparatus of the first aspect, an initial vessel, a sample vessel, and a fluid inlet tube.

### BRIEF DESCRIPTION OF THE DRAWINGS

One or more embodiments will now be described by way of example only with reference to the accompanying drawings in which:

FIGS. 1A-1C show an example embodiment of a fluid sampling apparatus of the present disclosure in a front view, an exploded view and a cross-sectional exploded view, respectively;

FIGS. 2A-2C show a fluid sampling apparatus with an actuation arm, where the components of the fluid sampling apparatus are provided in three different positions;

FIG. 3 shows a diagrammatic representation of how an example the fluid sampling apparatus can be used to obtain both initial fluid samples and desirable fluid samples;

FIG. 4 shows an example embodiment of a method of obtaining a fluid sample using a fluid sampling apparatus; and

FIG. 5 shows an example kit of parts including a fluid sampling apparatus.

### DETAILED DESCRIPTION

Fluid sampling apparatuses are designed to draw a desired amount of fluid from a reservoir of fluid. In the present disclosure, bodily fluids of humans or animals may be primarily referred to, however, devices such as those disclosed herein may have applications in sampling fluids from other sources. In particular, the fluids may be liquids stored in liquid reservoirs. The present disclosure provides a plurality of embodiments of fluid sampling apparatuses which provide beneficial features.

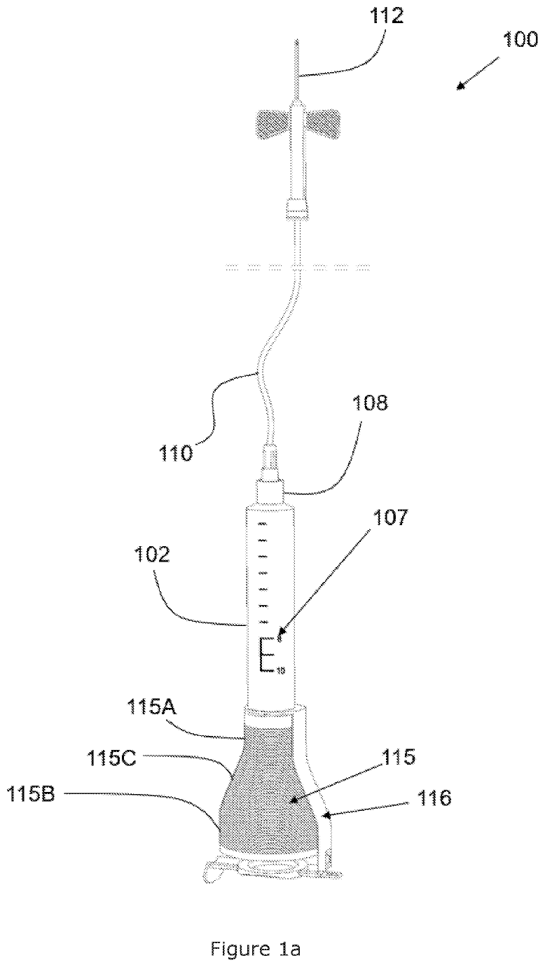

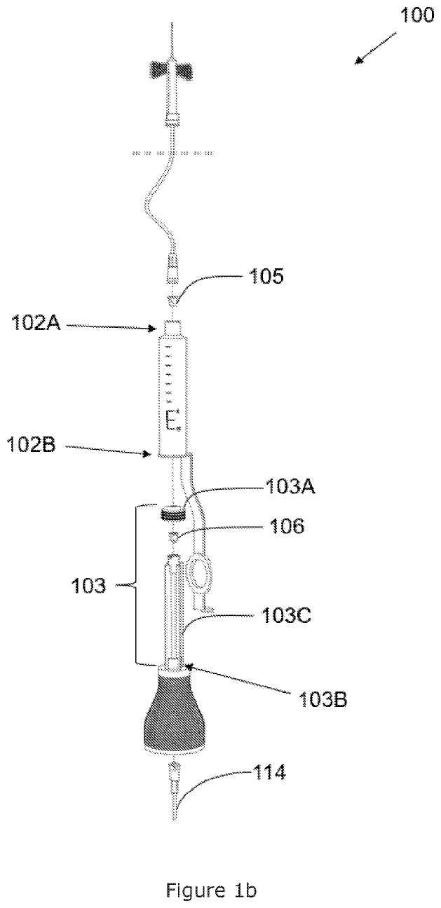

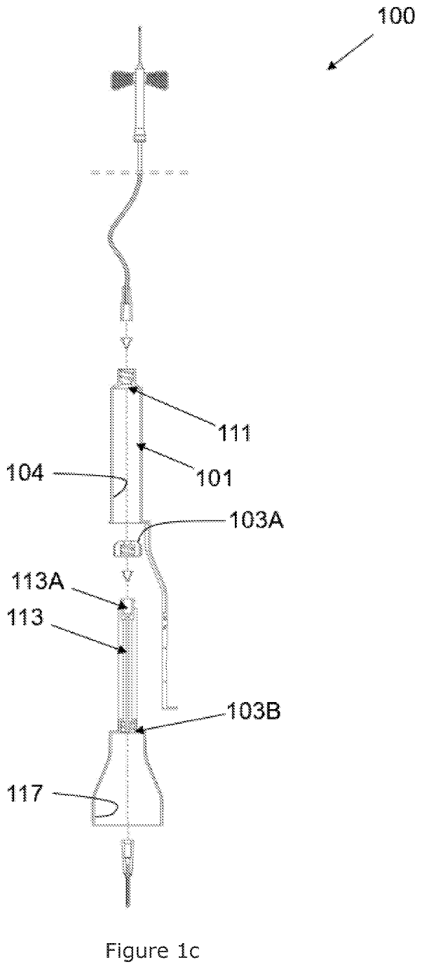

FIG. 1A shows an example embodiment of a fluid sampling apparatus 100 according to one or more embodiments of the present disclosure. While a plurality of components (constituent parts) of a fluid sampling apparatus 100 are shown in this embodiment, it will be appreciated that not all represented features are necessary components for providing an advantageous fluid sampling apparatus 100. Similarly, FIG. 1B shows the example embodiment of FIG. 1 in an exploded view such that several more components of the fluid sampling apparatus 100 are visible. Yet further, FIG. 1C shows an example embodiment of a cross-sectional exploded view of the fluid sampling apparatus 100 of FIG. 1. Like-reference numerals will be used between FIGS. 1A-1C for ease of reference. Some reference numerals will not be repeated between figures in order to avoid crowding FIGS. 1B and 1C with additional reference numerals.

The fluid sampling apparatus 100 comprises a primary fluid chamber 101 (FIG. 1C) that is configured to receive a fluid therein. The fluid may be a bodily fluid such as blood, spinal fluid or another bodily fluid. In other embodiments, the fluid may be a non-bodily fluid. The features of the disclosed fluid sampling apparatus are not limited to only providing utility to bodily fluids, although some embodiments may be particularly suited to this task.

The primary fluid chamber 101 is defined by a barrel 102 and a plunger 103. In particular, the primary fluid chamber 101 is defined by the inner walls 104 of the barrel 102 and by a first end of the plunger 103A. The first end of the plunger 103A may be referred to as the seat 103A of the plunger or the head 103A of the plunger. The plunger 103 may be slidably movable within the barrel 102 in order to vary the volume of the primary fluid chamber 101.

The barrel 102 may comprise a hollow elongate body and a first end 102A at one end of the elongate body and a second end 102B at the opposing end of the elongate body.

The cross-section of the elongate body of the barrel 102 may typically be a circular cross-section, however, it will be appreciated that different cross-sectional shapes may also be possible. The internal walls 104 of the barrel will define the periphery of the primary fluid chamber 101.

In particular, the barrel 102 may act as the sidewalls and top of the primary fluid chamber 101 and a seat of the plunger 103A (the upper surface of the plunger which moves forwards and backwards through the barrel) may provide the base of the primary fluid chamber 101. The seat 103A of the plunger may be separable from the plunger 103 or it may be monolithic with the plunger 103.

The plunger 103 will be arranged such that it provides a seal to the desired fluid for the primary fluid chamber 101. For example, in one or more embodiments, the plunger 103 may be arranged and sized within and relative to the barrel 102 such that it provides a hermetic seal to the barrel 102. In one or more examples, the seal may not need to be hermetic and may only need to be sufficiently sealed to prevent the egress of the intended fluid from the primary fluid chamber. That is, the plunger 103 may provide for a leak-proof seal suitable for the intended fluid to be sampled. In some embodiments, it may only be the first end of the plunger 103A which is configured to provide for the seal to the barrel 102, as a suitable seal at or towards the first end of the plunger 103 may obviate the need for any further sealing along the length of the plunger 103. The sealing portion of the plunger, which may be the head of the plunger 103A, may be separable from the body 103C of the plunger and the upper portion of the body 103C of the plunger or the head 103A of the plunger may comprise space for a second one-way valve 106 to be stored therewithin. In other examples, the plunger head 103A may be monolithic with the second one-way valve 106. That is, the plunger head 103A may have the second one-way valve 106 built in. The plunger head may comprise one or more O-rings in order to provide an improved sealing between the plunger 103 and the barrel 102.

The barrel 102 may comprise at least one volume marking 107 configured to provide an indication of a volume of fluid stored in the primary fluid chamber 101. The volumetric markings 107 may be read by the alignment of the first end 103A of the plunger with the markings. This position of the plunger 103 relative to the volume markings 107 visible through the barrel 102. Volume markings 107 may provide one way of determining the volume of fluid drawn into the primary fluid chamber 101.

At the first end of the barrel 102, there may be a fluid inlet port 108 which provides for fluid communication between a coupled fluid inlet tube 110 and the primary fluid chamber 101 via a first one-way valve 105 positioned within the fluid inlet port 108. The second end 102B of the barrel may be substantially open such that it is configured to receive the plunger 103 therethrough. The first end 102A of the barrel may comprise a bevelled, chamfered or otherwise shaped portion 111 which provides for a narrowing of the primary fluid chamber 101 towards the first end 102A of the barrel. The fluid inlet port 108 may extend outwards from the shaped portion 111 of the barrel 102 and may be positioned substantially centrally to the cross-section of the barrel 102. In alternative embodiments, it may not be necessary for the fluid inlet port 108 to be positioned substantially centrally to the cross-section of the barrel 102.

The fluid inlet port 108 which couples to the fluid inlet tube 110 may be shaped to engage with and retain the fluid inlet tube 110. For example, the fluid inlet port 108 may comprise a cylindrical protrusion with a channel therethrough such that the fluid inlet tube 110 can extend over or within the cylindrical protrusion of the fluid inlet port 108 and be held thereto by a friction fit. In some examples, the cylindrical protrusion may be a luer lock fitting or a luer-slip fitting. Alternatively, the cylindrical protrusion may be a different type of fitting or adapter suitable for coupling the fluid inlet tube 110 to the fluid inlet port 108. It will be appreciated that the protrusion of the fluid inlet port 108 may not be cylindrical and, instead, may have a different shape. The fluid inlet port 108 may be monolithic with the rest of the barrel 102 or it may be a separate component which is coupled to the barrel 102 by way of any appropriate means.

Where a fluid inlet tube 110 is coupled to the fluid inlet port 108 at a second end of the fluid inlet tube 110, the fluid inlet tube 110 may comprise a butterfly needle 112 at its first end. The butterfly needle 112 may alternatively be referred to as a butterfly set 112 or a winged infusion set 112. It will be appreciated that other types of fluid inlet tube 110 may be used. A butterfly needle 112 may be an example of a body piercing member and the body piercing member may be any suitable apparatus for piercing a body (or other barrier) and drawing a fluid through itself into the fluid inlet tube 110.

The plunger 103 further comprises a body 103C that contains a plunger channel 113 therethrough that extends from the first end 103A of the plunger to the second end 103B of the plunger, wherein the second end 103B of the plunger is the surface of the plunger that opposes the first end 103A. In this way, the plunger 103 may comprise a channel 113 that extends substantially straight through the plunger 103. The plunger channel 113 provides for the flow of fluid from the primary fluid chamber 101 into a fluid vessel (not shown) coupled at the second end 103B of the plunger. The body 103C of the plunger may also be referred to as the stem 103C of the plunger. The second end 103B of the plunger may be referred to as the handle 103B of the plunger.

The first end 103A of the plunger may be bevelled, chamfered or otherwise shaped such that it corresponds to the bevelling, chamfering or otherwise shaping of the shaped end 111 of the first end of the barrel 102. This may allow for the first end 103A of the plunger to be received within the shaped end 111 of the first end 102A of the barrel such that, when the plunger 103 is fully inserted into the barrel 102, the volume of the primary fluid chamber 101 is zero, or substantially zero. By providing for such a mating arrangement of the first end 103A of the plunger with the shaped end 111 of the barrel, the fluid inlet port 108 and the first end of the plunger channel 113A may be placed in direct fluid communication such that fluid does not flow via the primary fluid chamber 101 to get to the plunger channel 113 when the plunger 103 is fully inserted into the barrel 102. It will be appreciated that other approaches may be utilised in order to provide for direct fluid communication between the fluid inlet port 108 and the plunger channel 113 when the plunger 103 is in a particular position within the barrel 102.

The first one-way valve 105 may be configured to provide for fluid flow from a coupled fluid inlet tube 110, through the fluid inlet port 108 and into the primary fluid chamber 101. The first one-way valve 105 may further be configured to prevent the flow of fluid from the primary fluid chamber 101 back into the fluid inlet tube 110. The first one-way valve 105 may be configured to provide for, and restrict, these fluid flows as described due to the hydraulic resistance of the one-way valve 105 being selected to have an appropriate resistance. More particularly, the first one-way valve 105 may have a forward hydraulic resistance and a different backward (or reverse) hydraulic resistance wherein the backward hydraulic resistance is greater than the forward hydraulic resistance.

For example, when the plunger 103 is fully inserted into the barrel 102, or substantially fully inserted into the barrel 102, such that a fluid connection is provided directly between the fluid inlet port 108 and the plunger channel 113, the forward hydraulic resistance may be sufficient that no fluid will flow into the plunger channel 113 until a primary sample vessel (hereinafter referred to as a sample vessel) or an initial sample vessel (hereinafter referred to as an initial vessel for conciseness) is coupled to the second end 103B of the plunger.

The sample vessel may be used for obtaining primary samples which are samples which are substantially or entirely uncontaminated with barrier particles corresponding to a barrier that is pierced in order to allow for fluid to be drawn by the fluid sampling apparatus 100. For example, the barrier may be skin where the fluid sampling apparatus 100 is a blood sampling apparatus. It will be appreciated here that the term “primary” is used to refer to main or foremost samples that are desired and does not limit primary samples to the first samples or even a single sample. One may take several primary samples after the initial contaminated sample has been taken. The initial vessel may be used for obtaining initial samples which may be expected to comprise contamination from barrier particles or other surface contaminants that are present in the first samples of fluid which are drawn after an initial barrier is pierced.

The sample vessel may comprise a different cross-section to the initial vessel. For example, the cross-sectional area of the main body of the sample vessel may be greater than the cross-sectional area of the main body of the initial vessel. The diameter of the cross-section of the sample vessel may be greater than the diameter of the cross-section of the initial vessel. In further examples, the shape of the cross-section of the sample vessel may be different to the shape of the cross-section of the initial vessel.

A typical sample vessel or initial vessel may have a negative pressure and this negative pressure may provide for sufficient force to overcome the forward hydraulic resistance of the first one-way valve 105. In other examples, the first one-way valve 105 may not resist the flow of fluid into the plunger channel 113 without the presence of a sample vessel or initial vessel.

The second one-way valve 106 may be configured to provide for or restrict fluid flow from the fluid inlet port 108 or from the primary fluid chamber 101 (depending on the position of the plunger 103) from flowing into the plunger channel 113 depending on the hydraulic resistance of the selected second one-way valve 106. For example, when the plunger 103 is moved such that the volume of the primary chamber 101 is increased, the second one-way valve 106 may have a sufficient hydraulic resistance that fluid will not flow out of the primary fluid chamber 101 and through the plunger channel 113 unless a sample vessel having a negative pressure is coupled to the second end 103B of the plunger. This may be particularly advantageous, as it may allow for a fluid sample to be drawn into the primary fluid chamber 101 without having the sample vessel coupled to the second end 103B of the plunger. This may assist in being able to make reliable volume measurements prior to the transfer from the primary fluid chamber 101 into the sample vessel.

The backwards hydraulic pressure, which may be referred to as reverse hydraulic pressure, of each of the first one-way valve 105 and the second one-way valve 106 may be sufficient to prevent fluid from flowing in a reverse direction under normal operating condition of the fluid sampling apparatus 100. The reverse direction may be considered to be, for example, from the plunger channel 113 into one of the primary fluid chamber 101 or from the primary fluid chamber 101 into the fluid inlet tube 110.

The first and second one-way valves 105, 106 may be any type of valve having suitable forward and backward hydraulic resistances to provide for the functionality as described and, fundamentally, to only allow for fluid flow in a single direction during the operation of the fluid sampling apparatus 100 under normal conditions. For example, a one-way valve may be a check valve, a non-return valve, a reflux valve, a retention valve, a foot valve or another type of one-way valve that only allows for fluid to flow through it in a single direction under normal conditions.

The second end 103B of the plunger is configured to engage with a sample vessel for receiving a fluid from the plunger channel 113. The second end 103B of the plunger may be configured to provide for engagement with the sample vessel by way of its size and structure or by way of additional components fixed to it. For example, the second end 103B of the plunger may be sized and shaped to receive a sample vessel thereover and provide for a friction fit between the sample vessel and the second end 103B of the plunger.

In other embodiments, the second end 103B of the plunger may comprise a piercing member 114 which is configured to pierce the sample vessel in order to direct the flow of fluid from the plunger channel 113 to the sample vessel. In particular, the piercing member 114 may be configured to pierce a cap of the sample vessel and the cap of the initial vessel, where used. It will be appreciated that piercing members 114 may be replaceable and, as such, a fluid sampling apparatus 100 may be provided without a piercing member 114 in place but the second end 103B of the plunger may be configured to engage with a piercing member prior to use.

Yet further, the second end 103B of the plunger may be configured to be interchangeably couplable between each of an initial vessel and a sample vessel. Providing for interchangeable coupling between an initial vessel and a sample vessel may comprise being sized suitably to receive either an initial vessel or a sample vessel. The second end 103B of the plunger may be further configured to be interchangeably coupled between an initial vessel and a sample vessel by having one or both of a sample vessel adapter 115 or an initial vessel retention arm 116. The sample vessel adapter 115 and initial vessel retention arm 116 are discussed in further detail below.

Typically, an initial vessel and a sample vessel will have different volumes and, more particularly, the main body of an initial vessel will typically have a smaller cross-sectional diameter than the main body of a sample vessel. A typical initial vessel may be any suitable blood collection tube for biochemical analysis. For example, the initial vessel may be a vacutainer tube such as a BD Vacutainer. A typical initial vessel may comprise dimensions of substantially 15 mm×15 mm×82 mm, for example. A typical sample vessel where blood is the fluid of interest may be a blood culture bottle such as a BD BACTEC™ blood culture media or bioMérieux BacT/ALERT® blood culture bottle. Alternatively, the blood culture bottle may be a VersaTREK system or another type of blood culture bottle. A sample vessel, such as a blood culture vessel, may have dimensions of substantially 39 mm×39 mm×147 mm or 28 mm×28 mm×119 mm. In general, an initial vessel may comprise a smaller cross-sectional diameter than a sample vessel. For example, the initial vessel may have a diameter of 25 mm or less, 20 mm or less or 15 mm or less. Further, a sample vessel may comprise a diameter of 30 mm or more, 35 mm or more or 40 mm or more.

Initial fluid samples, such as those taken from the human or animal body, drawn by a fluid sampling apparatus 100 may comprise impurities and contaminants which are unsuitable for or may interfere with certain tests or analysis. As such, it is useful to be able to be able to separate an initial sample without needing to remove the body piercing member 112 (such as a needle, in the case of a body fluid sampling apparatus 100) or otherwise dismantle the fluid sampling apparatus 100. Such interruptions increase the risk of introducing or re-introducing undesirable contaminants. The provision of the plunger channel 113 having a volume substantially equal to that desired, or otherwise typical, for an initial sample may provide for an effective to way to ensure that the proper minimum quantity of initial sample has been extracted prior to taking a sample for measurement or analysis. Providing the second end of the plunger that is configured to be interchangeably coupled between each of an initial vessel and a sample vessel further improves the provision of a fluid sampling apparatus 100 configured to allow for the removal of such an initial sample.

In some embodiments, the initial sample may be discarded after being drawn in some cases. In other examples, however, the initial sample may not be discarded. For example, the initial sample may be used for certain types of analysis or tests that do not require sterility of the sample; such tests may include, for example, chemical testing. It can be particularly difficult to draw blood from some patients and, as such, being able to make use of every bit of blood drawn is very beneficial. Providing for a system which allows the initial sample to be drawn into a container (the initial vessel) which can be used for testing may be particularly advantageous for this reason.

The second end 103B of the plunger may further comprise a sample vessel adapter 115. The sample vessel adapter 115 may be configured to at least partially engage with a sample vessel when the sample vessel is fluidly coupled to the primary fluid chamber 101 via the plunger channel 113. For example, the sample vessel adapter 115 may comprise a frustoconical portion 115C. That is, the diameter of the cross-section of the sample vessel adapter 115 may steadily vary from a first diameter at a first end 115A of the sample vessel adapter to a second diameter at a second end 115B of the sample vessel adapter wherein the second diameter is larger than the first diameter.

The first diameter at the first end 115A of the sample vessel adapter may be substantially similar to that of the plunger 103 or the barrel 102. For example, the first diameter may be within 5%, 10% or 20% of the diameter of the barrel 102. The first end 115A of the sample vessel adapter may be sized to fit over the second end 103B of the plunger and be retained on the second end 103B of the plunger by a friction fit or by another fastening means. In one or more alternative embodiments, the sample vessel adapter 115 may be monolithic with the plunger 103.

The second diameter at the second end 115B of the sample vessel adapter may be from 40-80% greater than the diameter of the barrel 102. In particular, the second end 115B of the sample vessel adapter may be sized to extend over but still physically engage with a typical sample vessel. Thus, an inner diameter of the second end 115B of the sample vessel adapter may be substantially the same as that of a standard sample vessel such that the sample vessel adapter 115 can provide for physical engagement with a standard sample vessel. In particular, the inner walls 117 of the sample vessel adapter may be sized to engage with a sample vessel. Further, the frustoconical portion 115C of the sample vessel adapter may provide a helpful shape for gripping by a user.

Where the second end 103B of the plunger comprises a piercing member 114, the piercing member 114 may be sized such that it does not extend beyond a second end of the sample vessel adapter 115. In this way, the sample vessel adapter 115 may provide protection to the piercing member 114 to prevent it from getting knocked, damaged or contaminated by it touching another object or person. Further, the sample vessel adapter 115 may, in the same way, provide for protection to users from getting stabbed by the piercing member 114 accidentally by effectively providing a wide protective sheath to the piercing member 114.

The plunger 103 may be moveable between a first position and a second position wherein, in the first position, the fluid inlet port 108 is in fluid communication with the plunger channel 113 such that fluid does not flow into the primary fluid chamber 101. When the plunger 103 is in the first position, the volume of the primary fluid chamber 101 may be zero or substantially zero. In other embodiments, the primary fluid chamber 101 may have a non-zero volume when the plunger is in the first position, but the configuration of the fluid inlet port 108 and the plunger channel 113 may be such that there is no fluid flow into the primary fluid chamber 101 when the plunger 103 is in the first position.

In the second position, the primary fluid chamber 101 has a predefined volume and the fluid inlet port 108 is in fluid communication with the primary fluid chamber 101 such that fluid flowing into the fluid inlet port 108 enters the primary fluid chamber 101. The predefined volume may be the target volume for the fluid for which the fluid sampling apparatus is designed. For example, where the target fluid is blood, the volume may be 5-20 ml of blood such as 10 ml.

Movement of the plunger 103 from the first position to the second position may comprise sliding the plunger 103 through the barrel 102 without the first end 103A of the plunger leaving the body of the barrel 102.

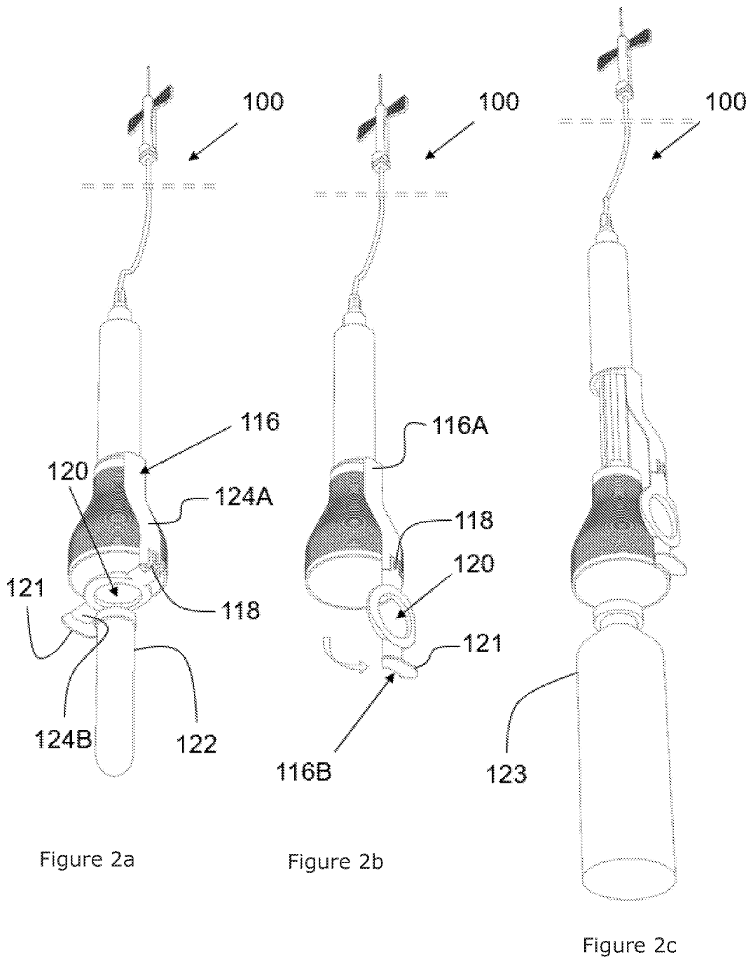

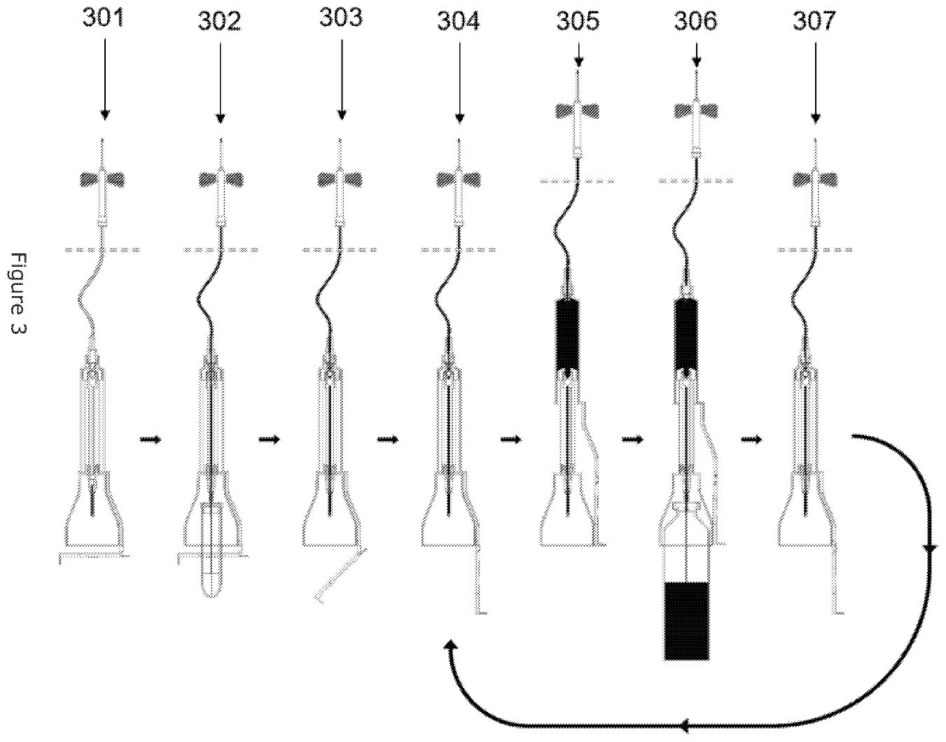

FIGS. 2A-2C show the example fluid sampling apparatus 100 having an actuation arm 116 wherein the actuation arm 116 is also a sample vessel retention arm. The features of FIG. 2 are substantially similar to those of FIG. 1 and, as such, like reference numerals will be used for consistency. Further, reference numerals will not be repeated features that have already been described in detail.

The fluid sampling apparatus 100 may comprise the actuation arm 116 coupled at a first end 116A of the actuation arm to the barrel 102. The second, opposite, end 116B of the actuation arm is configured to extend beyond the second end of the other components of the fluid sampling apparatus 100 when the plunger 103 is in the first position. When the plunger 103 is in the second position, the second end 116B of the actuation arm is in line with the second end of the other components of the fluid sampling apparatus 100.

For example, the second end 116B of the actuation arm may extend beyond the second end 103B of the plunger when the plunger 103 is in the first position and the second end 116B of the actuation arm may be in line with the second end 103B of the plunger when the plunger 103 is in the second position.

In another example, where the fluid sampling apparatus 100 comprises a sample vessel adapter 115 (such as the embodiments shown in FIGS. 2A-2C), the second end 116B of the actuation arm may extend beyond the second end 115B of the sample vessel adapter when the plunger 103 is in the first position. When the plunger 103 is in the second position, the second end 116B actuation arm is in line with the second end 115B of the sample vessel adapter. For example, FIG. 2B shows the second end 116B of the actuation arm extending beyond the second end 115B of the sample vessel adapter when the plunger 103 is in the first position. FIG. 2C shows the second end 16B of the actuation arm as in line with the second end 115B of the sample vessel adapter when the plunger 103 is in the second position.

The actuation arm 116 may provide for improved ease of measurement of the volume of fluid drawn into the primary fluid chamber 101. More specifically, the actuation arm 116 may provide for improved ease of measurement of the volume of the primary fluid chamber 101 which, when fluid is drawn correctly, will directly correspond to the volume of the drawn fluid. For example, when seeking to draw a predetermined volume of fluid using the fluid sampling apparatus 100, a user may hold the fluid sampling apparatus 100 by the sample vessel adapter 115 or the plunger 103 and place the actuation arm 116 against a solid surface, such as a table, wall or the user's chest, and then pull the plunger 103 or sample vessel adapter 115 until it is in line with the second end 116B of the actuation arm. In this way, a correct (predetermined) volume of fluid can be drawn using the fluid sampling apparatus 100 without the need to read volume markings 107 from the barrel 102. This also allows a healthcare professional (the user in the case that the apparatus is used for drawing bodily fluids) to focus their attention on their patient instead of on the volumetric markings. This may allow for improved sample volume accuracy and repeatably accurate sample volumes. In one or more embodiments, it may be particularly intended that the user holds the sample vessel adapter 115. In such embodiments, a protective lip may be provided between the first end of the sample vessel adapter and the second end 103B of the plunger in order to prevent, or reduce the likelihood, of a user gripping the plunger. Gripping by the plunger 103 may increase the likelihood of sample contamination as the gripped portion of the plunger 103 re-enters the barrel 102.

While the example of FIGS. 2A-2C show an actuation arm 116 comprising a hinged joint 118 and, an initial vessel retention aperture 120 and a load bearing surface 121, it will be appreciated that none of these features are essential to the actuation arm 116 providing its function of allowing the collection of repeated collection of reliable fluid volumes.

The actuation arm 116 may comprise a load bearing surface 121 at the second end 116B of the actuation arm wherein the load bearing surface 121 extends perpendicularly to the length of the actuation arm 116 such that the load bearing surface 121 is configured to provide a surface against which force can be distributed in order to reduce the pressure on a surface pressing against the second end 116B of the actuation arm. That, is, the actuation arm 116 may comprise an elongate body which extends from its first end 116A to its second end 116B. The cross-sectional area of the load bearing surface 121 may be a multiple of the cross-sectional area of the body of the actuation arm 116. For example, the cross-sectional area of the load-bearing surface 121 may be twice, three times, four times, five, ten, or another multiple of the cross-sectional area of the body of the actuation arm 116. If the second end 116B of the actuation arm does not have a load bearing surface 121, then pushing against the second end 116B of the actuation arm may apply a large pressure against the force-applying surface. If the force-applying surface is part of the human body, then this may cause pain to the user or piercing of the user's skin. By providing a load bearing surface 121 which extends perpendicularly to the length of the actuation arm (defined as the direction from the first end 116A of the actuation arm to the second end 116B of the actuation arm), the force applied to the second end 116B of the actuation arm is spread over a larger area, thereby making it more comfortable for a user applying force with a part of their body.

The fluid sampling apparatus 100 may further comprise an initial vessel retention arm 116 wherein the initial vessel retention arm 116 is movable between a first position (FIG. 2A) and a second position (FIG. 2B or 2C). In the examples of FIGS. 2A-2C, the actuation arm 116 and the initial vessel retention arm 116 are the same member. It will be appreciated that one of an actuation arm 116 and a retention arm 116 may be provided without the other, if desired. Alternatively, two separate members could be provided wherein one provides the functionality of the actuation arm and the other provides the functionality of the retention arm.

The initial vessel retention arm 116 further comprises an initial vessel retention aperture 120 in which an initial vessel 122 can be retained. For example, the aperture may be sized or otherwise configured to provide mechanical support to an initial vessel 122.

In the first position, at least a portion of the initial vessel retention arm 116 may be configured to substantially align the initial vessel retention aperture 120 with a piercing member 114 coupled to the second end 103B of the plunger such that an initial vessel 122 retained within the initial vessel retention aperture 120 is pierced by the piercing member 114. That is, when the initial vessel retention arm 116 is in its first position, the initial vessel retention aperture 120 is positioned such that fluid flowing out of the plunger channel 113 via a piercing member 114 would fall through the aperture 120 in the absence of an initial vessel 122. Blood flow absent a vessel should not occur in normal usage but this description is provided in order to give an example of the alignment of the initial vessel retention aperture 120.

When the initial vessel retention arm 116 is in the second position, the initial vessel retention aperture 120 may not be in line with the piercing member 114 such that the initial vessel retention arm 116 does not impede the coupling of a sample vessel 123 to the piercing member 114. That is, initial vessel retention arm 116 may be configured to be moveable such that its position does not inhibit the coupling of a sample vessel 123 to the piercing member 114 at the second end 103B of the plunger when in the second position.

In one or more embodiments, the initial vessel retention aperture 120 may provide for loose retention of the initial vessel 122 such that it does not hold the initial vessel 122 in place alone but it provides for lateral support and guidance of the initial vessel 122. That is, the diameter of the initial vessel retention aperture 120 may be greater than the diameter of a typical initial vessel 122. More particularly, the diameter of the initial vessel retention aperture 120 may be greater than the diameter of a typical initial vessel 122 but small than that of a typical sample vessel 123. The initial vessel retention aperture 120 may ensure the initial vessel 122 can be properly engaged with the piercing member 114 at the second end 103B of the plunger without a user needing to look carefully to ensure the initial vessel 122 is aligned with the piercing member 114. In such an embodiment, it may be possible to insert the initial vessel 122 through the initial vessel retention aperture 120 from below the initial vessel retention aperture 120. The initial vessel retention arm 116 may be configured to be in the first position prior to use of the fluid sampling apparatus 100. By providing the initial vessel retention arm 120 in the first position, it blocks a user from, possibly by accident or force of habit, coupling a sample vessel to the second end 103B of the plunger. This may help to reduce the chance of one or both of wasting sample vessels 123 and sending potentially contaminated samples to the testing laboratory.

In other embodiments, the initial vessel retention aperture 120 may be configured such that an initial vessel 122 inserted into the initial vessel retention aperture 120 is held in place by the initial vessel retention aperture 120 as a result of the size and shape thereof relative to the initial vessel. For example, the diameter of the initial vessel retention aperture 120 may be equal to or smaller than the diameter of at least a portion of a typical initial vessel 122; in this way, an initial vessel 122 may be retained within the initial vessel retention aperture 120.

In one or more embodiments, the initial vessel retention arm 116 may comprise a first initial vessel retention sub-arm 124A and a second initial vessel retention sub-arm 124B. That is, the initial vessel retention arm 116 may comprise a first portion 124A and a second portion 124B wherein each portion 124A, 124B (sub-arm) is a member (such as an elongate member). The first initial vessel retention sub-arm 124A may be coupled to one of the barrel 102, the plunger 103 or the sample vessel adapter 115. Where the first initial vessel retention sub-arm 124A does not provide any functionality other than the holding of an initial vessel 122, it may not matter to which component the initial vessel retention arm 116 is coupled as long as it is able to move from supporting an initial vessel 122 to allowing a sample vessel 123 to be coupled to the second end 103B of the plunger. The second initial vessel retention sub-arm 124B may comprise the initial vessel retention aperture 120. The second initial vessel retention sub-arm 124B may be moveable between the first position and the second position of the initial vessel retention arm 116. The first initial vessel retention sub-arm 124A may be coupled to the second initial vessel retention sub-arm 124B by an articulated joint 118. The articulated joint 118 may be any suitable type of articulated joint 118 that allows for the movement of the second sub-arm 124B from the first position to the second position. For example, the articulated joint 118 may be provided by a hinge. In the first position, the second initial vessel retention sub-arm 124B may extend perpendicularly to the first initial vessel retention sub-arm 124A. In this configuration, the first initial vessel retention sub-arm 124A may extend substantially parallel to the barrel 102 and the plunger 103. Where the fluid sampling apparatus 100 comprises a sample vessel adapter 115, one or both of the first initial vessel retention sub-arm 124A and the second initial vessel retention sub-arm 124B may comprise an inflected portion that allows the initial vessel retention arm 116 to substantially match the shape of the sample vessel adapter 115. This may allow for the initial vessel retention arm 116 to track along or near the outer edge of the sample vessel adapter 115 when the plunger is in the first position in order to provide for a compact design. When the second initial vessel retention sub-arm 124B is in its second position, the second initial vessel retention sub-arm 124B may extend parallelly to the first initial vessel retention sub-arm 124A. For example, the second initial vessel retention sub-arm 124B may extend away from the first initial vessel retention sub-arm 124A such that the initial vessel retention arm 116 as a whole extends substantially continuously in a single direction from its first end 116A to its second end 116B. In another example, the second initial vessel retention sub-arm 124B may fold backwards such that it is both parallel and adjacent to the first initial vessel retention sub-arm 124A.

As previously discussed, in one or more embodiments, such as that shown in FIGS. 2A-2C, the initial vessel retention arm 116 may also be the actuation arm 116. That is, the actuation arm 116 may be configured to provide for both the functionality of allowing the acquisition of a predetermined volume of fluid by way of its alignment with a component of the fluid sampling apparatus (such as alignment with the second end 115B of the sample vessel adapter) and the functionality of the initial vessel retention arm 116 to support an initial vessel 122 in a first position and provide for space for a sample vessel 123 in a second position. In embodiments where the initial vessel retention arm 116 and the actuation arm 116 are the same component, the arm will be coupled to the barrel 102. Thus, the actuation arm 116 may be movable between a first position and a second position wherein the actuation arm 116 comprises an initial vessel retention aperture 120 in which an initial vessel 122 can be retained.

When the actuation arm 116 is in the first position, at least a portion of the actuation arm 116 may be configured to substantially align the initial vessel retention aperture 120 with a piercing member 114 of the second end 103B of the plunger such that an initial vessel 122 retained within the initial vessel retention aperture 120 is pierced by the piercing member 114.

When the actuation arm 116 is in the second position, the actuation arm 116 may not be in line with the piercing member such that the actuation arm 116 does not impede the coupling of a sample vessel 123 to the piercing member 114. Further, when in the second position, the actuation arm may provide for moving the plunger 103 from its first position to its second position within the barrel 102 in order to accurately and repeatedly obtain the predetermined volume of fluid.

Alternatively, a separate initial vessel retention arm and actuation arm may be provided.

FIG. 3 depicts an example process for using a fluid sampling apparatus according to the one or more embodiments of the present disclosure. FIG. 3 shows 7 stages which can represent the operation of a fluid sampling apparatus. It will be appreciated that some of these stages are optional stages, as they incorporate the use of non-essential features of the disclosure. Further, reference numerals for individual components will not be provided in relation to these figures. The example fluid sampling apparatus of FIG. 3 shows an embodiment that corresponds to those of FIGS. 1A-2C and, as such, reference to these figures can be made for individual component reference numerals.

In the first stage 301, a fluid sampling apparatus 100 is provided as described with reference to FIGS. 1A-1C and 2A-2C. The fluid sampling apparatus 100 is configured to have the plunger 103 in its first position such that the fluid inlet port 108 and the plunger channel 113 are in direct fluid communication via both of the first one-way valve 105 and the second one-way valve 106. An actuation arm 116 comprises a first sub-arm 124A and a second sub-arm 124B wherein the second sub-arm 124B comprises an initial vessel retention aperture 120. In this stage, the second sub-arm 124B is in its first position such that, when an initial vessel 122 is inserted into the initial vessel retention aperture 120, the initial vessel 122 will be retained therein and the initial vessel 122 will be aligned with a piercing member 114 coupled to the second end 103B of the plunger such that the piercing member 114 pierces the initial vessel 122. Providing the second sub-arm 124B in the second position from the outset also prevents accidental coupling of a sample bottle 123 prior to taking an initial sample in an initial vessel 122. The fluid sampling apparatus 100 at stage one 301 is also coupled to a fluid inlet tube 110 at a second end of the fluid inlet tube and the fluid inlet tube 110 is further coupled to a butterfly needle 112 at its first end.

In the second stage 302, an initial vessel 122 is inserted into and retained within the initial vessel retention aperture 120. The piercing member 114 pierces a cap of the initial vessel 122. The cap of the initial vessel may be, for example, a bromobutyl rubber stopper or a different appropriate cap. Because, in this described embodiment, the initial vessel 122 comprises a negative pressure prior its cap being pierced, fluid (which may be blood, for example) flows from the butterfly needle 112, through the fluid inlet tube 110 to the fluid sampling apparatus 100. At the fluid sampling apparatus 100, the fluid flows into the fluid inlet port 108 and is able to pass through the one-way valve 105 as a result of the negative pressure of the pierced initial vessel 122. Because the plunger 103 is in the first position, the fluid flows from the fluid inlet port 108 directly into the plunger channel 113 via the second one-way valve 106. The negative pressure of the initial vessel 122 may allow for the fluid to overcome the forward hydraulic resistance of the one-way valves. The fluid flows through the plunger channel 113 and the piercing member 114 at the second end 103B of the plunger into the initial vessel 122. This second stage 302 may be performed after insertion of the butterfly needle 112 into, for example, a patient or into another fluid reservoir. Where this fluid is the first fluid drawn from a fluid reservoir, it may comprise contaminants such as skin particles or, more generally, particles of the barrier which is pierced in order to access the fluid reservoir. The amount of fluid drawn in this stage may depend on the negative pressure within the initial vessel 122 such that fluid will continue to flow into the initial vessel 122 until a pressure equalisation or the initial vessel 122 is detached from the piercing member 114 or until a user detaches the sample vessel 122. In embodiments wherein blood is the fluid of interest, the initial sample may be 1.5 ml-4 ml in volume, for example.

The inner diameter of the plunger channel 113 may be substantially equal to the inner diameter of a typical fluid inlet tube 110 which is to be coupled to the fluid sampling apparatus 100. The inner diameter of the plunger channel 113 may also be substantially equal to the inner diameter of the fluid inlet port 108. By providing for substantially equal inner diameters, one may avoid or reduce the formation of air bubbles that may otherwise form in the plunger channel 113.

In the third stage 303, the initial vessel 122 is removed from the initial vessel retention aperture 120 and the second sub-arm is shown moving from its first position into its second position. In this embodiment the first sub-arm and the second sub-arm are hingably coupled. It may be that the removal of the negatively pressurised initial vessel 122 removes a force that was previously allowing fluid to overcome the forward hydraulic resistance of the first and second one-way valves 105, 106 and, as such, fluid may be stopped, or substantially stopped, from flowing out of the piercing member 114 at the second end of the plunger 103B. The piercing member 114 may be covered by a rubber cover that gets pushed back when a sample or initial vessel 122, 123 is introduced. This may prevent leakage from the needle when no sample vessel 122, 123 is in place.

In the fourth stage 304, the second sub-arm 124B is fully moved into the second position such that it does not impede the coupling of a sample vessel 123 to the second end 103B of the plunger. At this stage, the plunger 103 is still in the first position.

In the fifth stage 305, the plunger 103 is moved from its first position to its second position such that the predetermined volume of fluid is drawn into the primary fluid chamber 101 through the first one-way valve 105. The movement of the plunger 103 from the first position to the second position creates an adjustable negative pressure that allows the fluid to overcome the forward hydraulic resistance of the first one-way valve 105 such that the fluid enters the primary fluid chamber 101. Since there is not a negative pressure in the plunger channel 113, fluid does not flow from the primary fluid chamber 101 into the plunger channel 113. As can be seen in the figure, in the second position of the plunger 103, the second end 116B of the actuation arm is in line with the second end of the sample vessel adapter 115B. In this way, it is ensured that the correct predetermined volume is obtained. Further, a user may move the plunger 103 from the first position into the second position by holding the fluid sampling apparatus 100 by the sample vessel adapter 115 or the plunger 103, placing the actuation arm 116 against a surface, and pulling the plunger 103 or sample vessel adapter 115 downwards towards the second end of the actuation arm 116. Thus, this provides a fluid sampling apparatus 100 that can advantageously be operated accurately with a single hand.

In the sixth stage 306, a sample vessel 123 is coupled to the piercing member 114 at the second end 103B of the plunger. Coupling the sample vessel 123 to the piercing member 114 involves partially inserting the sample vessel 123 into the sample vessel adapter 115 such that at least a portion of the side walls of the sample vessel engage with the inner walls 117 of the sample vessel adapter. In particular, the inner walls 117 at the second end 115B of the sample vessel adapter may engage with the side walls of the sample vessel. In one or more embodiments, a first end, or a portion of the first end, of the sample vessel 123 may engage with a portion of the inner walls 117 of a frustoconical portion 115C of the sample vessel adapter. Any engagement between the sample vessel 123 and the sample vessel adapter 115 may provide for enhanced stability during use. A negative pressure within the sample vessel 123 may provide for some fluid flow from the primary fluid chamber 101 into the sample vessel 123.

In order to ensure the full volume of fluid stored in the primary fluid chamber 101 is transferred to the sample vessel 123, the plunger 103 may be moved from the second position back to the first position. The reverse hydraulic resistance of the first one-way valve 105 may prevent fluid from the primary chamber 101 from flowing back into the fluid inlet tube 110 during this process and, instead, the lower forward hydraulic resistance of the second one-way valve 106 may allow for fluid flow from the primary fluid chamber 101 into the sample vessel 123. Again, the fluid sampling apparatus 100 may be easy to operate with a single hand by, this time, holding the barrel 102 in a single hand, placing the sample vessel 123 against a surface and pulling downwards towards the surface. This may cause the barrel 102 to move relative to the plunger 103 such that the plunger 103 returns to the first position.

In the seventh stage 307, the plunger 103 has returned to the first position and the sample vessel 123 can be removed. As is evident in FIG. 3, the seventh stage 307 may be identical to, or substantially similar to, the fourth stage 304. As such, after the seventh stage 307 has been completed, the fluid sampling apparatus 100 can be operated to repeat the fifth through seventh stages 305, 306, 307 in order to acquire additional fluid samples. This may be particularly beneficial where more than a single sample of fluid is required. This solution provides the advantage that the fluid sampling apparatus 100 does not need to be disconnected from fluid inlet tube 110 or any other components (apart from a sample vessel 123) in order to repeatedly take a plurality of samples. Providing a fluid sampling apparatus 100 which allows for this approach further means that additional initial samples using additional initial vessels 122 do not need to be taken in order to take multiple fluid samples. This saves on wasted materials and costs related to initial vessels 122, wasted time required to take additional initial samples and wasted fluid.

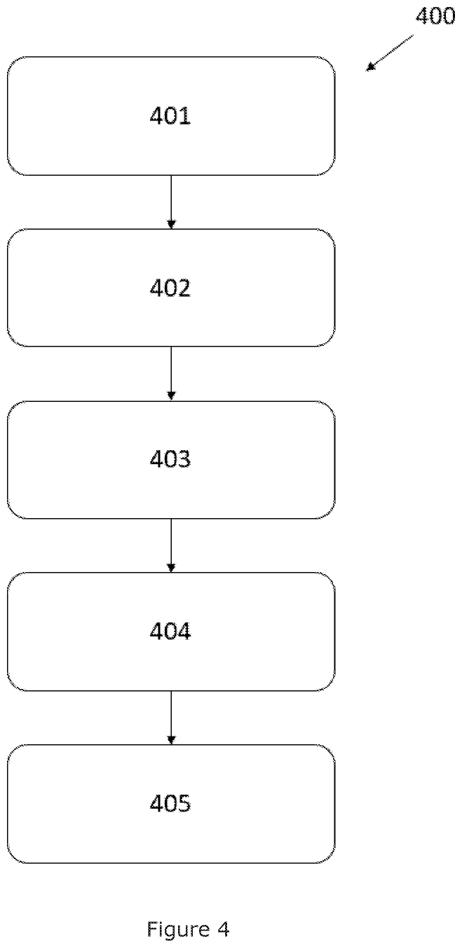

FIG. 4 shows an example method of using a fluid sampling apparatus of the present disclosure according to one or more embodiments of the present disclosure. The method comprises a step of coupling 401 the fluid inlet port to a fluid inlet tube at a second end of the fluid inlet tube. The fluid inlet tube is configured to receive a fluid at a first end of the fluid inlet tube. For example, the fluid may be provided via a body piercing member such as a needle or from another source or interface. For example, an intravenous (IV) catheter or peripheral venous catheter may be placed in a patient's arm and used to draw blood for tests. It may not be necessary to specifically pierce a body for some types of fluid that may be sampled. The method further comprises coupling 402 an initial vessel to the second end of the plunger such that fluid flows from the fluid inlet tube through the fluid sampling apparatus via the first and second one-way valves and into the initial vessel. The method further comprises removing 403 the initial vessel. Yet further, the method comprises moving 404 the plunger within the barrel such that the volume of the primary fluid chamber is increased, thereby creating a negative pressure in the primary fluid chamber and causing fluid to flow from the fluid inlet tube through the first one-way valve into the primary fluid chamber. Finally, the method may comprise coupling 405 a sample vessel to the second end of the plunger such that fluid flows from the primary fluid chamber into the sample vessel via the second fluid inlet valve.

In one or more embodiments, the method further comprises repeating the step of moving the plunger within the barrel such that the volume of the primary fluid chamber is increased and coupling a sample vessel to the second end of the plunger such that fluid flows from the primary fluid chamber into the vessel. In particular, coupling a sample vessel may comprise coupling a new sample vessel when the step is repeated. In this way, multiple samples can be collected without a need to disassemble or disconnect the fluid sampling apparatus. In other examples, a user may not need to move the plunger from the second position back into the first position, as the negative pressure exerted by the sample vessel may be sufficient to draw the fluid from the primary fluid chamber into the sample vessel by itself and, as a result, cause the plunger to move from the second position into the first position. This pressure and movement of the plunger may further prevent any additional fluid from being drawn through the first one-way valve. Once the plunger has returned to the first position, the first one-way valve may once more permit fluid to flow from the fluid inlet tube into the plunger channel. The method may further comprise the step of removing the sample vessel from the second end of the plunger once the plunger has returned to the first position.

The method of moving the plunger within the barrel such that the volume of the primary fluid chamber is increased may particularly comprise moving the barrel from the plunger from the first position to the second position. It will be appreciated herein the moving the barrel may particularly relate to moving the barrel relative to the plunger and that this may comprise moving the plunger relative to the barrel.

The step of coupling an initial vessel to the second end of the plunger may comprise moving the initial vessel retention arm into the first position and retaining the initial vessel within the initial vessel retention aperture. The step of removing the initial vessel may comprise removing the initial vessel retention arm from the initial vessel retention aperture and moving the initial vessel retention arm into the second position.

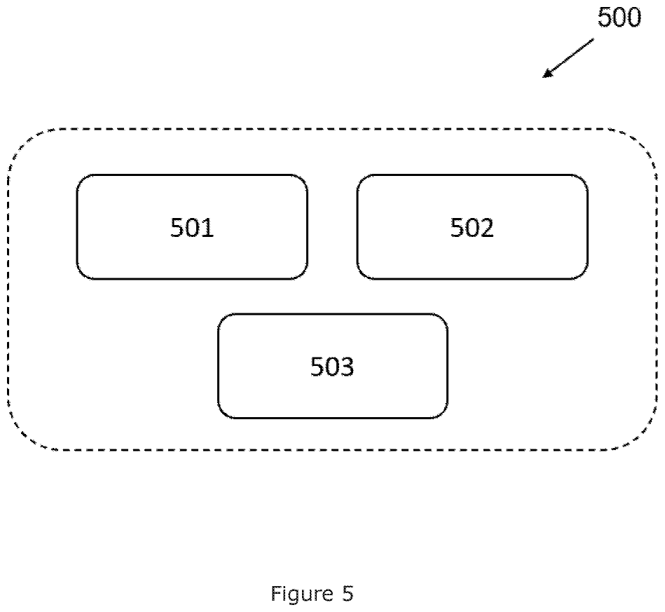

FIG. 5 shows an example kit of parts 500 comprising a fluid sampling apparatus 501, a fluid inlet tube 502, and a sample vessel 503. The kit of parts may further comprise an initial vessel. The kit of parts may yet further comprise a butterfly needle.

1. A fluid sampling apparatus comprising:
a primary fluid chamber configured to receive a fluid therein, the primary fluid chamber defined by a barrel and a plunger, wherein the plunger is arranged at least partially within the barrel such that the movement of the plunger through the barrel varies the volume of the primary fluid chamber, wherein:
a first end of the barrel comprises a fluid inlet port wherein the fluid inlet port is couplable to a fluid inlet tube, wherein the fluid inlet port includes a first one-way valve and wherein the fluid inlet port and the first one-way valve are configured to provide for fluid communication between a coupled fluid inlet tube and the primary fluid chamber;
the plunger comprises a plunger channel therethrough including a second one-way valve, the plunger channel and second one-way valve are configured to provide for fluid communication between the primary fluid chamber at a first end of the plunger and a second end of the plunger wherein the first end of the plunger defines an inner surface of the primary fluid chamber and the second end of the plunger opposes the first end of the plunger; and
wherein the second end of the plunger is configured to engage with a sample vessel for receiving a fluid from the plunger channel.

2. The fluid sampling apparatus of claim 1 wherein the second end of the plunger is configured to be interchangeably couplable between each of an initial vessel and a sample vessel.

3. The fluid sampling apparatus of claim 1 wherein the plunger is moveable between a first position and a second position wherein, in the first position, the fluid inlet port is in fluid communication with the plunger channel such that fluid does not flow into the primary fluid chamber and in the second position, the primary fluid chamber has a predefined volume and the fluid inlet port is in fluid communication with the primary fluid chamber such that fluid flowing into the fluid inlet port enters the primary fluid chamber.

4. The fluid sampling apparatus of claim 3 further comprising a sample vessel adapter arranged at the second end of the plunger configured to at least partially engage with the sample vessel when the sample vessel is fluidly coupled to the primary fluid chamber via the plunger channel.

5. The fluid sampling apparatus of claim 2 wherein the second end of the plunger further comprises a piercing member which is configured to pierce a cap of a sample vessel to direct the flow of fluid from the plunger channel to the sample vessel.

6. The fluid sampling apparatus of claim 4 comprising an actuation arm coupled at a first end of the actuation arm to the barrel and wherein a second, opposite, end of the actuation arm extends beyond the second end of the sample vessel adapter when the plunger is in the first position and when the plunger is in the second position, the second end of the actuation arm is in line with the second end of the sample vessel adapter.

7. The fluid sampling apparatus of claim 6 wherein the second end of the actuation arm comprises a load bearing surface at the second end of the actuation arm wherein the load bearing surface extends perpendicularly to the length of the actuation arm such that the load bearing surface is configured to provide a surface against which force can be distributed in order to reduce a pressure on a surface pressing against the second end of the actuation arm.

8. The fluid sampling apparatus of claim 2 further comprising an initial vessel retention arm wherein the initial vessel retention arm is movable between a first position and a second position wherein the initial vessel retention arm comprises an initial vessel retention aperture in which an initial vessel can be retained and wherein:
when the initial vessel retention arm is in the first position, at least a portion of the initial vessel retention arm is configured to substantially align the initial vessel retention aperture with the piercing member such that an initial vessel retained within the initial vessel retention aperture is pierced by the piercing member and
when the initial vessel retention arm is in the second position, the initial vessel retention aperture is not in line with the piercing member such that the initial vessel retention arm does not impede the coupling of a sample vessel to the piercing member.

9. The fluid sampling apparatus of claim 8 wherein the initial vessel retention arm comprises:
a first initial vessel retention sub-arm, wherein the first initial vessel retentions sub-arm is coupled to one of the barrel, the plunger or the sample vessel adapter; and
a second initial vessel retention sub-arm wherein the second initial vessel retentions sub-arm comprises the initial vessel retention aperture, and
wherein the first initial vessel retention sub-arm is coupled to the second initial vessel retention sub-arm by an articulated joint and wherein, in its first position, the second initial vessel retention sub-arm extends perpendicularly to the first initial vessel retention sub-arm.

10. The fluid sampling apparatus of claim 9 wherein in the second position, the second initial vessel retention sub-arm extends parallelly to the first initial vessel retention sub-arm

11. The fluid sampling apparatus of claim 8 wherein the first initial vessel retention sub-arm is coupled to the barrel and the initial vessel retention sub-arm is the actuation arm.

12. A method of extracting a fluid using a fluid sampling apparatus of claim 1, the method comprising:
coupling the fluid inlet port to a fluid inlet tube at a second end of the fluid inlet tube and wherein the fluid inlet tube is configured to receive a fluid at a first end of the fluid inlet tube;
coupling an initial vessel to the second end of the plunger such that fluid flows from the fluid inlet tube through the fluid sampling apparatus via the first and second one-way valves and into the initial vessel;
removing the initial vessel;
moving the plunger within the barrel such that the volume of the primary fluid chamber is increased, thereby creating a negative pressure in the primary fluid chamber and causing fluid to flow from the fluid inlet tube through the first one-way valve into the primary fluid chamber;
coupling a sample vessel to the second end of the plunger such that fluid flows from the primary fluid chamber into the sample vessel via the second fluid inlet valve.

13. The method of claim 12 using the fluid sampling apparatus of claim 3 wherein moving the plunger within the barrel such that the volume of the primary fluid chamber is increased comprises moving the barrel from the first position to the second position.

14. The method of claim 13 using the fluid sampling apparatus of claim 8 wherein:
the step of coupling an initial vessel to the second end of the plunger comprises moving the initial vessel retention arm into the first position and retaining the initial vessel within the initial vessel retention aperture; and
the step of removing the initial vessel comprises removing the initial vessel retention arm from the initial vessel retention aperture and moving the initial vessel retention arm into the second position.

15. A kit of parts comprising a fluid sampling apparatus of claim 1, an initial vessel, a sample vessel, and a fluid inlet tube.
